(19) United States
(12) Patent Application Publication
(10) Pub. No.: US 2021/0166384 A1
(43) Pub. Date: Jun. 3, 2021

(54) SYSTEM, METHOD AND COMPUTER-ACCESSIBLE MEDIUM FOR TISSUE FINGERPRINTING

(71) Applicant: The Trustees of Columbia University in the City of New York, New York, NY (US)

(72) Inventors: Sairam GEETHANATH, New York, NY (US); Rami VANGURI, New York, NY (US); John Thomas Vaughan, JR., New York, NY (US); Sachin R. JAMBAWALIKAR, New York, NY (US)

(21) Appl. No.: 17/170,273

(22) Filed: Feb. 8, 2021

Related U.S. Application Data
(63) Continuation of application No. PCT/US2019/046129, filed on Aug. 12, 2019.
(60) Provisional application No. 62/717,859, filed on Aug. 12, 2018.

(51) Int. Cl. G06T 7/00; G06N 3/08

(52) U.S. Cl. CPC G06T 7/0012; G06N 3/08; G06T 2207/20084; G06T 2207/20081; G06T 2207/10088

(57) ABSTRACT

Exemplary system, method, and computer-accessible medium for generating a magnetic resonance (MR) tissue fingerprint training network(s) can be provided, using which it is possible to, for example, receive first information related to a MR image(s) of a portion(s) of a phantom(s), partition the first information into a plurality of patches, and generate the MR tissue fingerprint training network(s) by applying a convolutional neural network(s) to the patches. The convolutional neural network(s) can be a fully convolutional neural network(s). Each of the patches can be a same size. The patches can be overlapping patches. A size of the patches can be 3×3 pixels. The MR tissue fingerprint training network can be generated based on float values for each of the patches.

### CROSS-REFERENCE TO RELATED APPLICATION(S)

This application relates to and claims priority from U.S. Patent Application No. 62/717,859, filed on Aug. 12, 2018, the entire disclosure of which is incorporated herein by reference.

### FIELD OF THE DISCLOSURE

The present disclosure relates generally to fingerprinting and more specifically, to exemplary embodiments of exemplary system, method and computer-accessible medium for tissue fingerprinting.

### BACKGROUND INFORMATION

MRI based imaging biomarkers, more specifically quantitative magnetic resonance (“qMR”) biomarkers, are increasingly being used to provide information used in the development of various therapeutic agents. These qMR markers have also shown to help improve MR imaging accuracy in clinical diagnostics of disease processes. qMR imaging seeks to measure fundamental magnetic resonance (“MR”) specific tissue properties. In contrast to routine clinical imaging, which employs difference in tissue properties (e.g., proton density, T1 relaxation and T2 relaxation) to generate image based on combination of these properties, for subjective or qualitative interpretation, qMR measures/quantifies these intrinsic MR properties (e.g., T1, T2 and PD) to produce quantitative maps of these parameters. Currently, the magnetic resonance imaging (“MRI”) research community is using quantitative mapping procedures to complement qualitative imaging. For quantitative imaging to reach its full potential, it can be beneficial to analyze measurements across systems as well as in a longitudinal direction. MR imaging systems are now expanding to include quantitative mapping of MR biomarkers (e.g., MR based iron quantification, fat quantification, elastography etc.) in addition to qualitative imaging. Although quantitative mapping of biomarkers can greatly increase the amount, reliability, and comparability of the data obtained from MR imaging, it requires careful standardization of protocols and validation of accuracy of measurements using phantoms (e.g., standard reference objects or calibration structures) to assess the repeatability and reproducibility of the measurements across MRI systems and time.

Magnetic Resonance Fingerprinting (“MRF”) is an accelerated acquisition-reconstruction method employed to simultaneously generate multiple parametric maps of T1, T2, Proton Density, and off-resonance. There has been an interest in developing MRF methods and applications ranging from multi-parametric biomarker determination to MR system characterization. The role of accelerated and quantitative MR imaging of pediatric patient populations is well established. MRF delivers both these desired features simultaneously, thereby providing a potential positive impact on clinical outcomes.

Thus, it may be beneficial to provide exemplary system, method, and computer-accessible medium for tissue fingerprinting which can overcome at least some of the deficiencies described herein above.

### SUMMARY OF EXEMPLARY EMBODIMENTS

Exemplary system, method, and computer-accessible medium for generating a magnetic resonance (“MR”) tissue fingerprint training network(s) can be provided, which can include, for example receiving first information related to a MR image(s) of a portion(s) of a phantom(s), partitioning the first information into a plurality of patches, generating the MR tissue fingerprint training network(s) by applying a convolutional neural network(s) to the patches. The convolutional neural network(s) can be a fully convolutional neural network(s). Each of the patches can be a same size. The patches can be overlapping patches. A size of the patches can be 3×3 pixels. The MR tissue fingerprint training network can be generated based on float values for each of the patches.

In certain exemplary embodiments of the present disclosure, the MR image(s) can be generated using a pseudorandom acquisition procedure. Parameters of the pseudorandom acquisition procedure can include, e.g., (i) a flip angle of a radiofrequency (RF) pulse, (ii) a phase of the RF pulse, (iii) a repetition time, (iv) an echo time, and/or (v) a sampling pattern. The pseudorandom acquisition procedure can be used to generate a MR signal that can include a property(ies), where the property(ies) can include (i) T1, (ii) a T2, (iii) a proton density, or (iv) an off-resonance. The property(ies) can be determined using a pattern recognition procedure. A dictionary can be generated including one or more MR signal evolutions using a Bloch equation procedure, and the property(ies) can be determined based on the dictionary. The property(ies) can be optimized using the CNN(s).

In further exemplary embodiments of the present disclosure, the CNN(s) can be trained using a further phantom(s). For example, the CNN(s) can be trained based on signal evolutions of neighboring voxels around a voxel of interest. In addition or alternatively, the CNN(s) can be trained based on the signal evolutions by concatenating the neighboring voxels around the voxel of interest. Further, in addition or alternatively, the CNN(s) can be trained based on magnetic resonance fingerprint (MRF) information. Channels in the MRF information can represent a temporal component of a radiofrequency signal. The CNN(s) can be a fully CNN(s).

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1A is an exemplary diagram of the signal evolution of a single pixel according to an exemplary embodiment of the present disclosure;

FIG. 1B is an exemplary diagram of a 3×3 patch for applying spatial constraints according to an exemplary embodiment of the present disclosure;

FIG. 1C is an exemplary diagram illustrating a stacked space-time patch according to an exemplary embodiment of the present disclosure;

FIG. 1D is an exemplary diagram of Simple, Generalized framework for Tissue Fingerprinting using fully convoluted networks according to an exemplary embodiment of the present disclosure;

FIG. 2 is an exemplary flow diagram of a method for generating a magnetic resonance tissue fingerprint training network according to an exemplary embodiment of the present disclosure; and

FIG. 3 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

### Exemplary MRF Acquisition and Reconstruction

### Exemplary Randomization of Acquisition Control Parameters

MRF can be used for data acquisition, and can use a pseudorandomized acquisition which can cause different materials to have a unique signal evolution. Parameters which can be pseudorandomized include flip angle (“FA”) and phase of RF pulses, repetition time (“TR”), echo time (“TE”) and sampling patterns. The result can be a signal evolution which can contain multiple material properties such as T1, T2, Proton Density, and off-resonance.

### Exemplary Simulation of Dictionary Using Bloch Equation and/or Extended Phase Graphs

Once the signal evolutions are acquired, the material properties can be derived through pattern recognition. This can be performed by constructing a dictionary of signal evolutions through the Bloch equation formalism of MR. The resulting dictionary can contain signal evolutions from many combinations of materials and system-related parameters. By matching the acquired signal evolution to one in the constructed dictionary, material properties can be derived. Typical reconstruction of MRF data can be performed with matching (e.g., using vector product) with the signal evolutions simulated from Bloch equations.

### Exemplary Generation of Acquisition Control Parameters Using Machine Learning

The optimization of acquisition parameters through deep learning methods can be used to enhance MRF reconstruction performance. This can include an adversarial generative network to learn Bloch simulators that produce unique signatures for different tissue types.

### Exemplary Deep Learning Based Reconstruction

The generation and usage of dictionaries via Bloch equation formalism MR can be computationally intensive, especially since the amount of samples generated in the dictionary can be related to the accuracy of the signal evolution matching. By directly synthesizing quantitative maps from signal evolutions using deep learning procedures, it can be possible to achieve a result with higher accuracy that can also be computationally efficient. The deep learning procedure can be built by “training” a network based on ground truth data, a dataset in which there can be signal evolutions and corresponding quantitative maps. This can be performed by applying an MRF acquisition procedure along with a ground truth acquisition. Once the network can be trained, it can be applied to new scans. This is known as “testing” the network.

### Exemplary Gold Standards, Ground Truth and In Vivo References

The ground truth data used for training a deep learning procedure can be related to the accuracy of the reconstruction. By developing a robust gold standard procedure training method, more accurate quantitative maps can be synthesized. Deep learning networks can be trained on phantoms optimized for corresponding quantitative maps. By training the deep learning procedure on phantoms which have known material properties within physiologically feasible ranges, the exemplary system, method, and computer-accessible medium can be more accurate when tested in vivo. This can be based on training a network on known quantitative values from a phantom instead of acquiring them in vivo. Thus, typical biases that can arise when acquiring in vivo quantitative maps can be avoided. This can also leverage the advantages of phase and phantom-vendor specified MR parameter range that can be validated through conventional scanning in comparison with site specific MRF implementation. This can be accomplished through a construction of the input data and output structure and the usage of a Fully Convolutional Network (“FCN”). The signal evolutions of N neighboring voxels around the voxel of interest can be concatenated and jointly estimated by the network (e.g., N+1 parametric values). This can provide for a generalized network that does not depend on the structure (e.g., anatomy) of the input data for training. At the same time, the network can learn that MR parameters can typically be spatially constrained to a finite range. This exemplary procedure for creating a sliding window around each voxel can further enhance robustness through increased averaging. The same, single network can be utilized to train multiple parametric values simultaneously.

Fully Convolutional Neural Networks have been used extensively for segmentation, where the input can be an image and the output can be a mask containing classes areas of the image belong to. More specifically, they have been successfully used for organ and lesion segmentation in medical imaging. These networks can be utilized by starting with temporal MRF data and using fully convolutional networks to synthesize quantitative maps. In other deep learning approaches, the data used to compute the weights of the network (e.g., known as a training set) and the data used to test a trained network (e.g., known as a validation set) can be both derived in vivo. The exemplary system, method, and computer-accessible medium, according to an exemplary embodiment of the present disclosure, can train the network purely on phantoms, where the ground truth quantitative map is well known. This can reduce systematic bias that can arise from training the network in vivo such as those specific to the trained anatomy and/or acquired system. For example, a fully convolutional network can be used. This architecture has been shown to work well for the task of segmentation where the task can be pixel-wise class prediction. This exemplary network can be modified to predict quantitative maps.

An exemplary FCN-8s architecture was used, which can have the highest resulting precision in the quantitative maps. There can be several modifications to a standard FCN-8s. First, instead of feeding the network whole 3-channel (“RBG”) images, the input can be MRF data where the channels can represent the temporal component. Thus, the input can have T (e.g., number of time samples in the signal evolution, 979 in the exemplary case) channels of input. Additionally, instead of feeding whole images, the network can be trained in a patch-wise fashion where the patch size can be a hyper-parameter currently set to 3×3. This means that the input can be (e.g., 979×3×3) Columns×Height×Width. There can be one patch constructed around every non-zero voxel. The MRF volume in XY space as well as through slices can be scanned, randomly, picking non-zero voxels and constructing patches around them. The exemplary network can be trained with 80% of the voxels and validated with the remaining 20%. Instead of outputting pixel-wise class labels (e.g., segmentation) it can be modified to output float values for the each patch (e.g., 9 values→3×3). Because of this, the exemplary network can be trained with mean square error loss instead of cross entropy, which can be beneficial for class based minimization. The exemplary system, method, and computer-accessible medium, according to an exemplary embodiment of the present disclosure, can include spatial constraints through the inclusion of the patch-wise input but also generalizes to multiple anatomies.

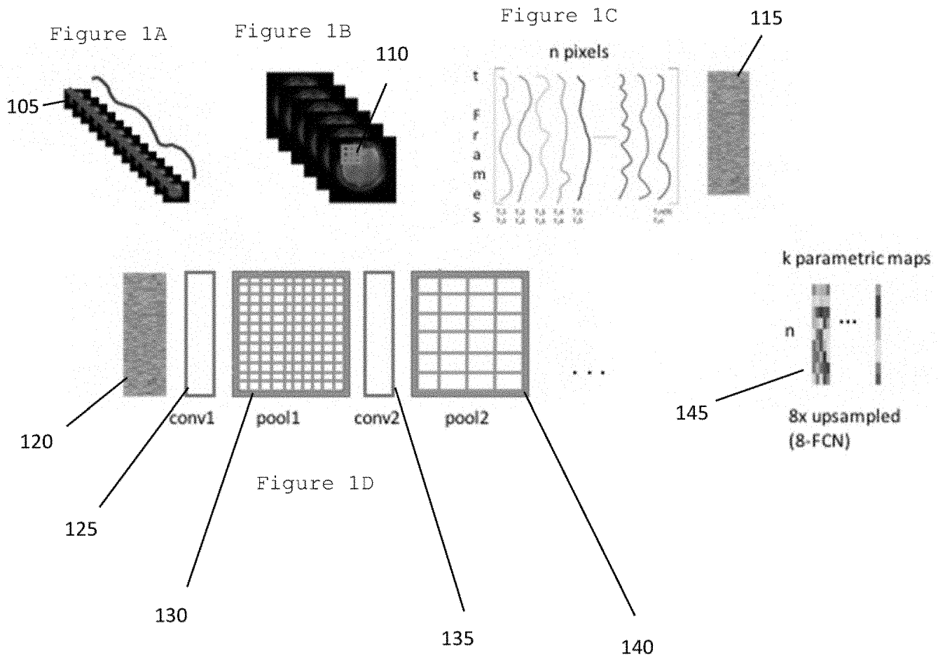

FIGS. 1A-1D show exemplary diagrams of exemplary workflow and implementation of the exemplary system, method, and computer-accessible medium according to an exemplary embodiment of the present disclosure. For example, FIG. 1A shows a diagram of an exemplary signal evolution of a single pixel 105. FIG. 1B shows an exemplary 3×3 patch 110 for applying spatial constraints. FIG. 1C shows a diagram of an exemplary stacked space-time patch 115, and FIG. 1D shows an exemplary implementation of SG-TiF using Fully Convolutional Networks (e.g., 8-FCN) producing k parametric maps of the n pixels. As illustrated in the diagram of FIG. 1D, a space-time patch 120 can be input into a convolutional neural network, which can include a first convolutional layer 125, a first pooling layer 130, a second convolutional layer 135, and a second pooling layer 140. The result can be one or more parametric maps 145.

The exemplary system, method, and computer-accessible medium, according to an exemplary embodiment of the present disclosure, can have the following benefits:
1) establish gold standard reference for MRF acquisition and reconstruction;
2) provide site-, magnetic field-, acquisition strategy-independent deep learning network;
3) facilitate a simple, quick, and robust MRF protocol implementation;
4) obtain increased precision and accuracy;
5) deliver high insensitivity to artifacts caused by acquisition;
6) directly reconstruct based on sensor data mapping to tissue parameters; and
7) estimate of tissue parameters not measured directly by MRI.

The exemplary system, method, and computer-accessible medium, according to an exemplary embodiment of the present disclosure, can include a scalable fingerprinting framework for tissue parameters measurable directly and indirectly with MRI—conductivity, temperature, etc. The exemplary system, method, and computer-accessible medium can include increased degrees of freedom in acquisition—randomization of trajectories, combination of sequence parameters for diverse contrasts such as, but not limited to, perfusion (e.g., contrast and non-contrast methods), diffusion, blood flow, etc. Drastic reduction in reconstruction computation times can be achieved, as compared to analytical methods (e.g., including multiple variants of Fourier transform) relying on gridding or iterative reconstruction for non-Cartesian and/or under-sampled acquisitions. The use of histopathological data as reference for fingerprints of pathology can facilitate training MRF sequences on stack of histopathological slides to understand the MRF signatures of such data; potentially to avoid biopsies in such anatomies.

### Exemplary Applications Rainbow

The exemplary system, method, and computer-accessible medium, according to an exemplary embodiment of the present disclosure, can be used for rapid comprehensive MRI exams, for example, pediatric neuroimaging, multi-parametric prostate imaging, whole body imaging oncology, diabetes studies inclusive of NASH, study of fat types such as brown, white and brite fat, etc. MR value driven protocols, for example, 5-minute stroke protocol, can be used as an alternate to EPImix, MAGIC, etc. The exemplary system, method, and computer-accessible medium can provide for a multi-scale, multi-modality image fusion, for example, rapid MR-PET exams for oncological applications, whole body metabolic disorders and neuro-psychiatric diseases such as AD, PD, MS and SZ. Atlas creation can be performed at higher field strengths to deliver increased information content at lower fields—synthesis of tissue parametric maps at higher fields can be utilized to train FCNs and employed for data generated from lower field strengths with appropriate correction factors that can be field dependent

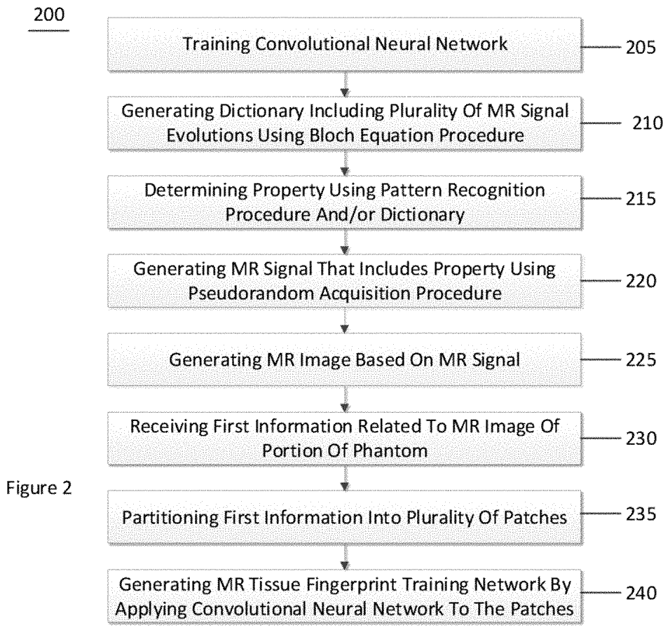

FIG. 2 shows an exemplary flow diagram of a method 200 for generating a magnetic resonance tissue fingerprint training network according to an exemplary embodiment of the present disclosure. For example, at procedure 205, a convolutional neural network (“CNN”) can be trained. At procedure 210, a dictionary can be generated, which can include a plurality of MR signal evolutions, using a Bloch equation procedure. At procedure 215, a property can be determined using a pattern recognition procedure and/or the dictionary. At procedure 220, a MR signal that includes the property can be generated using a pseudorandom acquisition procedure. At procedure 225, a MR image can be generated based on the MR signal, which can be received at procedure 230. Alternatively, the MR image may not be generated, and may only be received at procedure 230. At procedure 235, the first information can be partitioned into a plurality of patches. At procedure 240, the MR tissue fingerprint training network can be generated by applying the convolutional neural network to the patches.

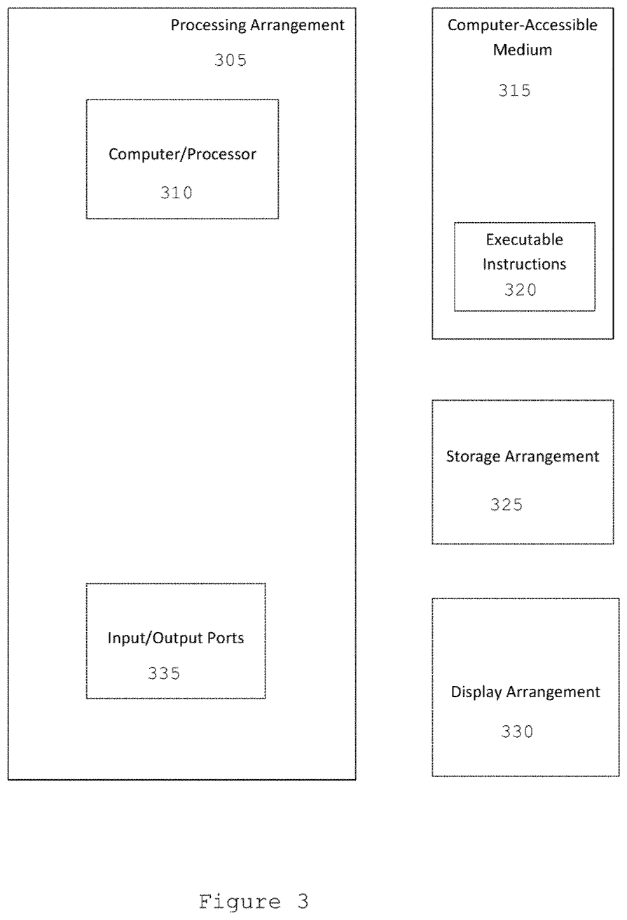

FIG. 3 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement or a hardware computing arrangement) 305. Such processing/computing arrangement 305 can be, for example entirely or a part of, or include, but not limited to, a computer/processor 310 that can include, for example one or more microprocessors, and use instructions stored on a computer-accessible medium (e.g., RAM, ROM, hard drive, or other storage device).

As shown in FIG. 3, for example a computer-accessible medium 315 (e.g., as described herein above, a storage device such as a hard disk, floppy disk, memory stick, CD-ROM, RAM, ROM, etc., or a collection thereof) can be provided (e.g., in communication with the processing arrangement 305). The computer-accessible medium 315 can contain executable instructions 320 thereon. In addition or alternatively, a storage arrangement 325 can be provided separately from the computer-accessible medium 315, which can provide the instructions to the processing arrangement 305 so as to configure the processing arrangement to execute certain exemplary procedures, processes, and methods, as described herein above, for example.

Further, the exemplary processing arrangement 305 can be provided with or include an input/output ports 335, which can include, for example a wired network, a wireless network, the internet, an intranet, a data collection probe, a sensor, etc. As shown in FIG. 3, the exemplary processing arrangement 305 can be in communication with an exemplary display arrangement 330, which, according to certain exemplary embodiments of the present disclosure, can be a touch-screen configured for inputting information to the processing arrangement in addition to outputting information from the processing arrangement, for example. Further, the exemplary display arrangement 330 and/or a storage arrangement 325 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference in their entireties.

[1] Ma, D., Gulani, V., Seiberlich, N., Liu, K., Sunshine, J. L., Duerk, J. L., & Griswold, M. A. (2013). Magnetic resonance fingerprinting. Nature. http://doi.org/10.1038/nature11971
[2] Oh, J., Cha, S., Aiken, A., Han, E. (2005). Quantitative apparent diffusion coefficients and T2 relaxation times in characterizing contrast enhancing brain tumors and regions of peritumoral edema. Resonance Imaging. Retrieved from http://onlinelibrary.wiley.com/doi/10.1002/jmri.20335/full
[3] Haskell, M., Cauley, S., & Wald, L. (2016). TArgeted Motion Estimation and Reduction (TAMER): Data consistency based motion mitigation using a reduced model joint optimization. Int Soc Magn Reson Med, Singapore.
[4] Assländer, J., Glaser, S., & Hennig, J. (2017). Pseudo Steady-State Free Precession for MR-Fingerprinting. Magnetic Resonance In. Retrieved from http://onlinelibrary.wiley.com/doi/10.1002/mrm.26202/full
[5] European Society of Radiology (ESR), E. S. of R. (2015). Magnetic Resonance Fingerprinting—a promising new approach to obtain standardized imaging biomarkers from MRI. Insights into Imaging, 6(2), 163-5. http://doi.org/10.1007/s13244-015-0403-3
[6] Hamilton, J., Jiang, Y., & Ma, D. (2016). Cardiac MR fingerprinting for T1 and T2 mapping in four heartbeats. Resonance. Retrieved from https://jcmr-online.biomedcentral.com/articles/10.1186/1532-429X-18-S1-W1
[7] MR fingerprinting for rapid quantitative abdominal imaging. (2016). Radiology. Retrieved from http://pubs.rsna.org/doi/abs/10.1148/radiol.2016152037
[8] Schmitt, P., Griswold, M., & Jakob, P. (2004). Inversion recovery TrueFISP: quantification of T1, T2, and spin density. Magnetic Resonance. Retrieved from http://onlinelibrary.wiley.com/doi/10.1002/mrm.20058/full
[9] MR fingerprinting using fast imaging with steady state precession (FISP) with spiral readout. (2015). Magnetic Resonance. Retrieved from http://onlinelibrary.wiley.com/doi/10.1002/mrm.25559/full
[10] Magnetic resonance fingerprinting. (2013). Nature. Retrieved from http://www.nature.com/nature/journal/v495/n7440/abs/nature11971.html
[11] Cao, X., Liao, C., Wang, Z., Chen, Y., & Ye, H. (2016). Robust sliding-window reconstruction for Accelerating the acquisition of MR fingerprinting. Magnetic. Retrieved from http://onlinelibrary.wiley.com/doi/10.1002/mrm.26521/full
[12] Balsiger, F., Shridhar, A., Chikop, S., Chandran, V., Scheidegger, O., Geethanath, S., & Reyes, M. (2018). Magnetic Resonance Fingerprinting Reconstruction via Spatiotemporal Convolutional Neural Networks. ArXiv Preprint ArXiv:1807.06356.

1. A non-transitory computer-accessible medium having stored thereon computer-executable instructions for generating at least one magnetic resonance (MR) tissue fingerprint training network, wherein, when a hardware computing arrangement executes the instructions, the hardware computing arrangement is configured to perform procedures comprising:
receiving first information related to at least one MR image of at least one portion of at least one phantom;
partitioning the first information into a plurality of patches; and
generating the at least one MR tissue fingerprint training network by applying at least one convolutional neural network (CNN) to the patches.

2. The computer-accessible medium of claim 1, wherein the at least one CNN is at least one fully convolutional neural network.

3. The computer-accessible medium of claim 1, wherein a size of each of the patches is the same.

4. The computer-accessible medium of claim 1, wherein the patches overlap one another.

5. The computer-accessible medium of claim 1, wherein the patches have the size of 3×3 pixels.

6. The computer-accessible medium of claim 1, wherein the hardware computing arrangement is further configured to generate the at least one MR tissue fingerprint training network based on float values for each of the patches.

7. The computer-accessible medium of claim 1, wherein the hardware computing arrangement is further configured to generate the at least one MR image using a pseudorandom acquisition procedure.

8. The computer-accessible medium of claim 7, wherein parameters of the pseudorandom acquisition procedure include at least one of (i) a flip angle of a radiofrequency (RF) pulse, (ii) a phase of the RF pulse, (iii) a repetition time, (iv) an echo time, or (v) a sampling pattern.

9. The computer-accessible medium of claim 7, wherein the hardware computing arrangement is further configured to utilize the pseudorandom acquisition procedure to generate at least one MR signal that includes at least one property, and wherein the at least one property includes at least one of (i) a T1, (ii) a T2, (iii) a proton density, or (iv) an off-resonance.

10. The computer-accessible medium of claim 9, wherein the hardware computing arrangement is further configured to determine the at least one property using a pattern recognition procedure.

11. The computer-accessible medium of claim 10, wherein the hardware computing arrangement is further configured to:
generate a dictionary that includes a plurality of MR signal evolutions using a Bloch equation procedure; and
determine the at least one property based on the dictionary.

12. The computer-accessible medium of claim 9, wherein the hardware computing arrangement is further configured to optimize the at least one property using the at least one CNN.

13. The computer-accessible medium of claim 1, wherein the hardware computing arrangement is further configured to train the at least one CNN using at least one further phantom.

14. The computer-accessible medium claim 1, wherein the hardware computing arrangement is configured to train the at least one CNN based on signal evolutions of neighboring voxels around a voxel of interest.

15. The computer-accessible medium of claim 14, wherein the computing arrangement is configured to train the at least one CNN based on the signal evolutions by concatenating the neighboring voxels around the voxel of interest.

16. The computer-accessible medium claim 1, wherein the hardware computing arrangement is configured to train the at least one CNN based on magnetic resonance fingerprint (MRF) information.

17. The computer-accessible medium of claim 16, wherein the MRF information includes channels which represent a temporal component of a radiofrequency (RF) signal.

18. The computer-accessible medium of claim 1, wherein the at least one CNN is at least one fully CNN.

19. A method for generating at least one magnetic resonance (MR) tissue fingerprint training network, comprising:
receiving first information related to at least one MR image of at least one portion of at least one phantom;
partitioning the first information into a plurality of patches; and
using a hardware computing arrangement, generating the at least one MR tissue fingerprint training network by applying at least one convolutional neural network to the patches.

20-36. (canceled)

37. A system for generating at least one magnetic resonance (MR) tissue fingerprint training network, comprising:
a hardware computing arrangement configured to:
receive first information related to at least one MR image of at least one portion of at least one phantom;
partition the first information into a plurality of patches; and
generate the at least one MR tissue fingerprint training network by applying at least one convolutional neural network to the patches.

38-54. (canceled)
